(19) United States
(12) Patent Application Publication
(10) Pub. No.: US 2026/0263591 A1
(43) Pub. Date: Sep. 10, 2026

(54) EDIBLE POWDERS WITH PROTEIN WITH STANDARDIZED AND REPRODUCIBLE ALLERGENICITY, AND METHODS FOR PREPARING THE SAME

(71) Applicant: N.V. NUTRICIA, Zoetermeer (NL)

(72) Inventors: Loret Sophia Michel Martinus KEULERS, Utrecht (NL); Leonardo CORNACCHIA, Utrecht (NL); Laura Susanne SLEUMER, Utrecht (NL); Catharina Theresia KNIPPING, Utrecht (NL)

(73) Assignee: N.V. NUTRICIA, Zoetermeer (NL)

(21) Appl. No.: 18/718,439

(22) PCT Filed: Dec. 16, 2022
(86) PCT No.: PCT/EP2022/086489

§ 371 Date: Jun. 11, 2024

(30) Foreign Application Priority Data
Dec. 16, 2021 (EP) 21215209.4

(51) Int. Cl. A61K 39/35; A23C 9/20; A61K 9/00; A61K 9/16

(52) U.S. Cl. CPC A61K 39/35; A23C 9/20; A61K 9/0053; A61K 9/1623; A61K 9/1694

(57) ABSTRACT

The invention concerns a process of preparing an allergenic, edible powder product suitable for introducing or reintroducing protein to a human subject allergic to the protein or at risk of becoming allergic to the protein, the process comprising: (a) providing a powder comprising protein and reducing sugar, wherein the powder comprises a milk protein comprising beta-lactoglobulin (BLG), preferably BLG-A; and (b) heating the powder of step (a) (homogeneously) within a heated, vibrating pipe, wherein the powder is heated at a temperature from 60 to 150° C., preferably from 70 to 120° C., more preferably from 70 to 100° C., wherein the relative humidity (RH) within the pipe is controlled below 80%, to obtain a powder wherein the protein has been glycated with the reducing sugar; (c) optionally adding any heat-sensitive components and/or bulking agents to the powder after step (b), (d) packaging the powder, to obtain a packaged amount of an allergenic, edible powder: (i) having a (predetermined) antigenicity reduced at least 20% compared to the protein reference; and/or (ii) wherein the glycated protein has a (predetermined) degree of glycation of at least 5% expressed as the percentage of lysine residues that are blocked by the reducing sugar on BLG, preferably BLG-A, wherein the powder is preferably characterized by at least (ii).

### FIELD

The present invention relates to a process of preparing edible powders with a reproducible glycation degree and antigenicity for introducing or reintroducing allergenic proteins to a human subject allergic to the protein or at risk of becoming allergic to the protein. The invention also relates to edible powders and kits comprising a plurality of edible powders for introducing or reintroducing protein to a human subject allergic to the protein or at risk of becoming allergic to the protein.

### BACKGROUND

Milk and egg allergies are among the most common food allergies in childhood. In particular, cow's milk allergy and hen's egg allergy have a prevalence rate between 1 to 3%, within children. Cow's milk allergy, for example, can result in anaphylactic reactions, and may have long term implications on the growth and nutritional wellbeing of a child.

One approach towards reducing the occurrence of an allergic response, is the avoidance of ingestion of the food product, typically protein, in question, to which the subject is allergic. This approach requires strict control of the diet of the subject, often combined with supplementation of deficient nutrients, caused by the elimination of a protein source.

An alternative approach, which is often used in infant formulae, is to avoid the use of intact milk proteins and instead use extensively hydrolysed protein formulas (eHF) or amino based formulas (AAF); the protein epitopes, which cause the allergic response, have been destroyed prior to ingestion. These approaches relieve the allergic symptoms and prevent further allergic reactions while supporting the nutritional well-being of the patient.

None of the above approaches however treat the underlying conditions of the allergy. Accordingly, it is becoming more common to introduce or reintroduce proteins that are causing the allergic response (via specific immune modulation) through controlled allergen presentation.

WO2007/054587 discloses methods of prompting oral tolerance in an animal comprising administering glycated proteins to the animal. WO2007/054587 also discloses methods for producing a food product by subjecting proteins contained in the product to a solid-state glycation process.

G. P. Bosman et al. (Limited Lactosylation of Beta-Lactoglobulin from Cow's Milk Exerts Strong Influence on Antigenicity and Degranulation of Mast Cells, Nutrients 2021, 13, 2041) showed that lactosylation of beta-lactoglobulin following Maillard reaction decreases the antigenicity of the protein. The reduction of antigenicity was found to be lactosylation-dependent. Despite these findings, there remains a need for a product (range) with a reliable and reproducible antigenicity suitable for introducing or reintroducing food protein to allergic subjects.

In current clinical practice, several techniques are used to introduce or reintroduce offending food proteins. For example, oral immunotherapy (OIT) involves the consumption of incremental doses of an offending protein. Therapy is initiated by providing doses of protein that are below a given threshold level that would trigger reactions in an allergic patient. Subsequently, the dosage amount of protein is gradually increased over time, typically over a period of several months. Once a maintenance dose of the protein is achieved, the patient has to ingest the protein dose for a given period of time (typically ≥1 years, possibly indefinitely) to maintain a protected, desensitized state. A disadvantage of OIT is that a strict diet is required over a prolonged period of time which may impact on the quality of life of a patient.

A form of oral immunotherapy is the so-called baked milk challenge, which involves the introduction or reintroduction of incremental doses of cow's milk proteins, via baked milk containing foods e.g. cookies; muffins; and pancakes, back into the diet of a patient in a supervised medical setting. Baked milk is used as a product as the majority of children can already tolerate it before cow' milk. Building further on the baked milk challenge concept, guidelines have been developed which involve the use of what is colloquially known as milk ladders. Milk ladders are easy-to-follow instructions for a patient or doctor which allow gradual introduction or reintroduction of incremental doses of cow's milk protein antigenicity or type of milk protein via baked milk containing foods which have been heated to different temperatures. It is different from baked milk challenge since it does not only involve an increase in dose, but also uses different products that are decreasing in heat treatment when climbing the ladder. For example, foods at the bottom of the food ladder, which are used as a starting point for an allergic patient, comprise a small amount of well-baked milk. In contrast, foods at the top of the milk ladder comprise a large amount of moderately heated milk. Accordingly, tolerance to milk proteins is achieved by moderating the amount of milk protein administered and by moderating, via heat treatment, the antigenicity of the milk proteins. A baked milk challenge or milk ladder removes the need for unnecessary dietary restrictions and thereby increases the quality of life of a patient. A baked milk challenge or milk ladder may also enable identification of individuals developing a tolerance at an earlier stage of life.

An associated disadvantage of using a baked milk challenge or milk ladder is that it is difficult for an individual to moderate the tolerated dose of protein in a given baked food. For example, an individual starting at the bottom of the milk ladder with a food comprising a small amount of well-baked milk, may still experience an allergic response to the protein. Accordingly, the individual may therefore have to reduce the portion size until no allergic response is felt. With that, it is found difficult to reproduce the antigenicity levels accompanied with a known portion size. Baking variances can result in antigenicity level differences, for example the core could still be raw while the outside is burned. Differences in kitchen appliances add to the variation. Furthermore, reducing the amount of milk in a food recipe or increasing the baking temperature to provide a tolerated dosage amount are challenging. Also fussy eaters may not like to use the milk ladder or specific foods part of the milk ladder and also non-fussy children may start to suffer from taste fatigue if they need to consume the same food each time. Another drawback is that these products are not always perceived as being part of a healthy diet (e.g. cookies). Accordingly, obtaining a standardised antigenicity of a given baked food with a high compliance is difficult to achieve. In the art there is a need for product range with a well-defined and reproducible antigenicity and protocols for OIT which can be straightforwardly (re) adjusted to the person's need and allergic response.

### SUMMARY OF THE INVENTION

Firstly, the inventors assessed the antigenicity of existing successful baked milk challenges and ladders, and found that baked milk protein is introduced in these ladders in such a way that the antigenicity is gradually increased in time, starting with cookies baked at high temperature, ending with cow's milk. The inventors believe that most progress in terms of reducing allergy is achieved in the early stages, when introducing the human subject allergic to the protein or at risk of becoming allergic to the protein, to a powder with a standardized and reproducible low antigenicity; the inventors focused on producing powders to replace products of the milk ladder, for example cookies, muffins and yogurts.

With those findings in mind, the inventors have developed a process that enables the production of nutritional protein-containing powders with a reproducible level of antigenicity for introducing or reintroducing protein to a human subject allergic to the protein or at risk of becoming allergic to the protein. It was found that the antigenicity can indeed be manufactured to a pre-determined level, but it is key to heat the protein and a reducing sugar in dry form, by heating the powder homogeneously within a heated, vibrating pipe, to an extent that the protein and reduced sugar glycate. It was also found that a predetermined and reproducible glycation degree is obtained by the process of the invention, wherein the glycation degree is expressed as the percentage of lysine residues that are blocked by a reducing sugar. The reproducible control of antigenicity can thus conveniently be monitored in the product in terms of the glycation degree, while it may be more convenient to express the control in terms of antigenicity in the context of the therapeutic application itself. With the term ‘reproducible glycation degree’ it is understood that glycation degree can be controlled during manufacture, preferably within 15%, more preferably within 10%, even more preferably within 5%, and the glycation degree may be controlled to a pre-determined (pre-set) value, preferably within 15%, more preferably within 10%, even more preferably within 5%. Without wishing to be tied down to any theory, the inventors believe that due to glycation, the IgE binding spots may be blocked, which hinders an allergic reaction to take place.

The temperature and humidity conditions in the pipe are selected to avoid caking of the powder, thus achieving homogeneous and continuous heating of the powder. It was found key to carry out the heating step in dry form; while wet heating and wet glycation was also found to result in reduced protein antigenicity, the antigenicity in wet conditions could only be reduced to a limited extent, could not controlled and hence was not reproducible; it was found that heating for 2-4 hrs at 65-95° C. reduced antigenicity but it was not possible to reproduce the antigenicity in a standardized way; reference is for instance made to FIG. 1 in WO2007/054587.

With the possibility to manufacture allergenicity-tailored edible powders, more efficient oral immunotherapy programs and kits for use in OIT are developed. The powders are also in a format that addresses the problems associated with existing baked milk challenges and ladders as described above. The inventors developed a kit which is particularly suited in the early stages, with low, reproducible antigenicity powders replacing for example cookies or muffins which are part of existing milk ladders.

With the term ‘reproducible antigenicity’ it is understood that antigenicity can be controlled during manufacture, preferably within 10%, more preferably within 5%, and the antigenicity may be controlled to a pre-determined (pre-set) value, preferably within 10%, more preferably within 5%. Antigenicity describes the ability to be specifically recognized by the antibodies generated as a result of the immune response to the given substance; antigenicity can be determined using Enzyme-Linked ImmunoSorbent Assay (ELISA) which is sensitive and accurate. Comparing to the milk ladder, the (milk protein) antigenicity for 100 ml of commercial, ultra-high temperature (UHT) cow's skim milk (Milbona AF Deutschland GmbH) is set at 100, and the antigenicity levels of milk-protein based powders used throughout the description and claims are normalized to the antigenicity of the 100 ml UHT-treated cow's milk reference. The UHT treatment of the reference preferably involves heating for 2-5 seconds at 140-150° C., but can also be any other corresponding UHT-combination of time and temperature. The 100 ml UHT-treated cow's skim milk is equivalent to 3.47 gram milk protein. Alternatively, the antigenicity of 100 ml pasteurized cow's milk can be set at 100%, wherein the milk has preferably been pasteurized for 20 seconds at 72° C., or a corresponding combination of time and temperature to reach the same.

The nutritional compositions may be subjected to further processing or handling at the consumer's end, but preferably the consumer is cautioned to refrain from substantive heat treatment, i.e. subjecting the nutritional product at temperatures above 40° C.; higher temperatures may induce protein denaturation and affect antigenicity. The composition may be accompanied from instructions to refrain from heat treatment during the preparations of the product for consumption. Throughout the application and claims, the terms ‘nutritional protein-containing powder, ‘edible powder’ and ‘edible powder product’ are used interchangeably.

The nutritional, protein-containing compositions having a reproducible antigenicity may advantageously be used to introduce or reintroduce protein into a diet of an individual in a controlled way via a stepwise increase of antigenicity levels over time. The nutritional compositions may be used to replace or mimic the milk ladder. Since the product has a reproducible antigenicity, it is not affected by the consumer's handling of the composition, and addresses the issues associated with conventional milk ladders and baked milk challenges.

### BRIEF DESCRIPTION OF THE DRAWINGS

FIG. 1 is a side view of a lifting device suitable for carrying out step (b) in the manufacturing method according to the invention with the various conduits and injection orifices and for extracting the gases to be conveyed in the device. In a known manner, the vibrating helical lifting device for the transport of bulk products comprises at least one ramp or closed helical channel 2 winding around a central shaft 1 and a vibrating base 3 supporting the assembly, said vibrating base 3 comprising at least two unbalanced motors 4 arranged diametrically with respect to the vertical axis XX′ of the cylindrical barrel central 1 and mounted on flanges 7 integral with said vibrating base 3. Said helical channel 5 is fixed on said central shaft 1 by spacers 5. Said helical tube 2, in the form of a coil with separate turns, may have a developed length which may vary between one meter and several hundred meters: the pulverulent product is introduced at one end 8 of said coil 2 towards the lower part of the barrel 1 and advances upward in said tube 2 under the effect of the vibrations generated by the vibrating base 3 until reaching the other end of said tube from which it is extracted by outlet 9. Preheated water vapor is continuously introduced along said helical channel 2 through at least one orifice 10 of said channel, opening into a part heated to a given temperature, greater than 100° C., and said water vapor is extracted by another orifice 11 located in this same heated part 13.

FIG. 2 Amount of furosine (mol furosine per mol protein) in samples A1-C3, wherein A1, B1, and C1 correspond to 0 min, RT\hat{ }Trials 1-3; A2, B2 and C2 correspond to 9 min 80° C., 9 min 100° C. Trials 1-3; and A3, B3 and C3 correspond to 9 min 80° C., 18 min 100° C. Trials 1-3.

FIG. 3A Lactosylation degree of beta-lactoglobulin A (BLG-A) expressed as % of lysine residues blocked by lactose on BLG-A, in samples A1-C3, corresponding to sample A1-C3 in FIG. 2.

FIG. 3B Average lactosylation degree of BLG-A in triplicates, expressed as FIG. 3A.

### DETAILED DESCRIPTION

According to a first aspect of the invention there is provided a process of preparing an allergenic, edible powder product suitable for introducing or reintroducing protein to a human subject allergic to the protein or at risk of becoming allergic to the protein, the process comprising:
(a) providing a powder comprising protein and reducing sugar; and
(b) heating a powder homogeneously within a heated, vibrating pipe, to obtain a powder wherein the protein has been glycated with the reducing sugar;
(c) optionally adding any heat-sensitive components and/or bulking agents to the powder after step (b),
(d) packaging the powder,
   to obtain a packaged, allergenic, edible powder having a reproducible (and predetermined) reduced antigenicity. Preferably, the powder provided in step (a) comprises a milk protein comprising beta-lactoglobulin (BLG), preferably BLG-A, for instance whey protein, including sweet or acid whey. A combination of temperature and relative humidity within the pipe are selected to prevent the heated powder from caking. In a preferred embodiment, the powder has an antigenicity in the range of 0.1-50, more preferably from 0.1 to 20, more preferably from 0.2 to 10, most preferably 0.2-5, based on the protein reference. In a preferred embodiment, the powder as an antigenicity reduced at least 20%, more preferably at least 25% based on the protein reference. In a preferred embodiment, the powder has an antigenicity which corresponds to that of a cookie or muffin in the milk ladder.

Consequently, there is provided a process of preparing an allergenic, edible powder product suitable for introducing or reintroducing protein to a human subject allergic to the protein or at risk of becoming allergic to the protein, the process comprising:
(a) providing a powder comprising protein and reducing sugar, wherein the powder comprises a milk protein comprising beta-lactoglobulin (BLG), preferably BLG-A;
(b) heating the powder of step (a) homogeneously within a heated, vibrating pipe,
wherein the powder is heated at a temperature from 60 to 150° C., preferably from 70 to 120° C., more preferably from 70 to 100° C.,
wherein the relative humidity (RH) within the pipe is controlled below 80%,
to obtain a powder wherein the protein has been glycated with the reducing sugar;
(c) optionally adding any heat-sensitive components and/or bulking agents to the powder after step (b),
(d) packaging the powder,
   to obtain a packaged amount of an allergenic, edible powder wherein the glycated protein has a reproducible and predetermined glycation degree.

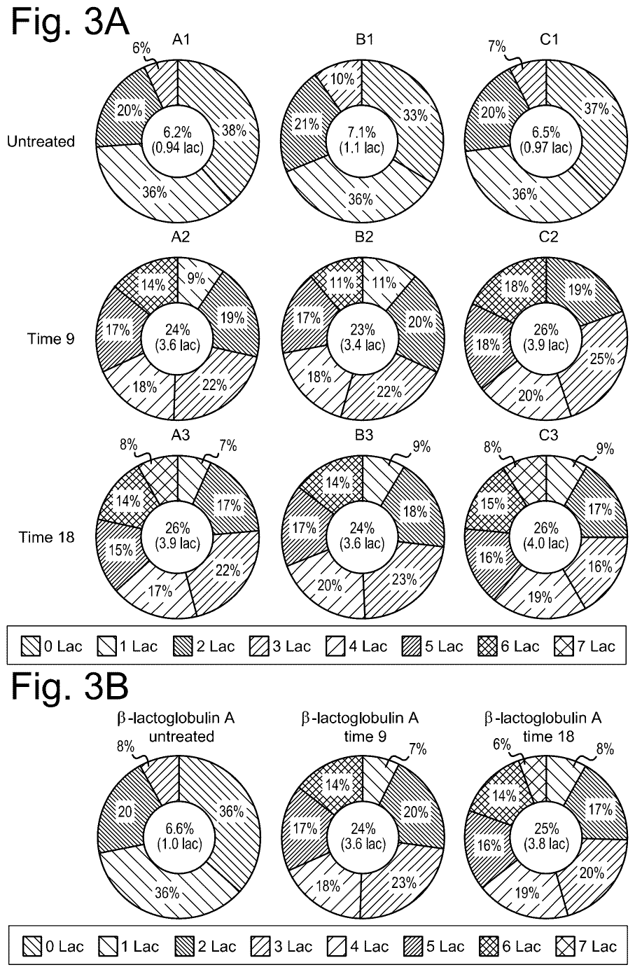

In the context of the invention, the degree of glycation is expressed as the percentage of lysine residues that are blocked by the reducing sugar in BLG, preferably BLG-A. BLG is one of the main components in milk whey protein and it is one of the main allergens. Given the abundance of BLG-A in milk protein, detection of glycation degree on BLG-A is more reliable than on other milk proteins, and the inventors also tested and found that the changes in glycation degree for BLG-A model the glycation degree for other proteins also present. For these reasons, BLG-A is selected as representative of glycation degree. Similar results in terms of glycation degree where obtained for BLG-B, hence the values of FIG. 3 are representative for BLG.

Preferably, the glycated milk protein has a degree of glycation of at least 5%, preferably at least 10%, more preferably at least 15%, most preferably 10-30% expressed as the percentage of lysine residues that are blocked by the reducing sugar on beta-lactoglobulin (BLG), preferably BLG-A. In one embodiment, the glycated milk protein has a degree of glycation of at least 20%, expressed as the percentage of lysine residues that are blocked by the reducing sugar on beta-lactoglobulin (BLG), preferably BLG-A. In a most preferred embodiment, the reducing sugar is lactose, in which case the above values for glycation degree apply to lactosylation degree.

The process may involve determining the desired, reduced antigenicity of the powder to be prepared by the process prior to (a), and selecting temperature, relative humidity and/or heating duration in heating step (b) to arrive at a reduced protein antigenicity that corresponds to the desired antigenicity. Advantageously, the process allows for only small error margins between the obtained and desired antigenicity of the powder, and from one to the next production process, which is preferably less than 10%, more preferably less than 5%. Such a product may advantageously be used in a ladder for oral immunotherapy as described further below.

The process according to the first aspect of the invention yields an allergenic edible powder comprising a glycated protein having a reproducible and predetermined glycation degree. Advantageously, the process allows for only small error margins between the obtained and desired glycation degree of the protein in the powder—and as shown in the examples thus implying a small error margin between the obtained and desired antigenicity of the protein in the powder—, and from one to the next production process, which is preferably less than 15%, more preferably less than 10%, even more preferably less than 5%. In a preferred embodiment, the glycated protein is milk protein, comprising beta-lactoglobulin (BLG), preferably BLG-A, for instance whey protein, including sweet or acid whey. The inventors surprisingly found that a very high glycation degree could be reached by the process of the invention. The inventors show that a lactosylation degree of up to 26% could be obtained in BLG-A (Example 2a and FIG. 3).

The process may involve the addition of a bulking agent such as maltrodextrin to the powder.

According to a second aspect of the invention there is provided a packaged edible powder comprising protein which has been glycated with a reducing sugar, wherein the powder has a reproducible reduced antigenicity, preferably in the range of 0.1-50, more preferably from 0.1 to 20, more preferably from 0.2 to 10, most preferably 0.2-5, based on the reference, in a predetermined portion (in weight), preferably obtained or obtainable by the process according to the first aspect of the invention. Alternatively, the antigenicity may correspond to that of a cookie or muffin in the milk ladder. The powder product preferably has a preset antigenicity. The powder has a reproducible antigenicity meaning that the actual and predetermined antigenicity preferably show less than 10% variation, preferably less than 5% variation. In a preferred embodiment, the powder may comprise a bulking agent such as maltrodextrin in order to ease consumer's handling of the powder. Additionally or alternatively, the packaged edible powder according to the second aspect of the invention comprises protein which has been glycated with a reducing sugar. Protein glycation serves as an indicator of antigenicity. Without wishing to be bound by a theory, the more a protein is glycated, the lower the antigenicity. This is confirmed by general knowledge in the art as evidenced by Bosman (2021) and WO2007/054587, and serves to characterize the powders in a simplified and straightforward yet reliable manner.

In a preferred embodiment, the glycated protein is milk protein, for instance whey protein, including sweet or acid whey, wherein the protein comprises beta-lactoglobulin (BLG), preferably BLG-A. Most preferably, the glycated protein is beta-lactoglobulin. Preferably, the glycated milk protein has a glycation degree of at least 5%, preferably at least 10%, more preferably at least 15%, most preferably 10-30% expressed as the percentage of lysine residues that are blocked by the reducing sugar on BLG, preferably BLG-A. In one embodiment, the glycated milk protein has a glycation degree of at least 20%. In any case, the powder has a reproducible glycation degree meaning that the actual and predetermined glycation degree preferably show less than 15% variation, more preferably less than 10% variation, even more preferably less than 5% variation.

Also, there is provided a kit of parts comprising: a) a plurality of a first allergenic, edible powder, having a first reduced reproducible antigenicity and/or a first increased reproducible glycation degree of the glycated protein comprised in the powder, and preferably accompanied from guidelines indicating the amount of individually packed first edible powder with a dose, for use in inducing oral immune tolerance to protein to a human subject being or at risk of becoming allergic to the protein; and/or for treating a human subject being or at risk of becoming allergic to the protein, wherein the protein to which the human subject is introduced or reintroduced in time with incremental, predetermined antigenicity. The protein comprised in the powder is a glycated milk protein comprising beta-lactoglobulin (BLG), preferably BLG-A, for instance whey protein, including sweet or acid whey. Preferably the protein is cow's milk protein and preferably the human subject is allergic or at risk of becoming allergic for cow's milk protein. In this embodiment, the first allergenic edible powder has a first increased and reproducible glycation degree of the glycated milk protein.

The reproducible antigenicity has allowed the inventors to develop a kit comprising a first plurality of edible powder compositions with a pre-determined, reproducible antigenicity per portion, which the consumer may use as part of an OIT program.

The kit preferably also comprises a second and optionally a third plurality of edible powder compositions with a pre-determined, reproducible antigenicity, wherein the antigenicity is increasing incrementally going from the first to the second to the optionally third plurality, wherein the kit is suitable for use in OIT, i.e. for use in introducing or reintroducing protein to a human subject allergic to the protein or at risk of becoming allergic to the protein. The skilled person will understand that as the antigenicity increases incrementally, the glycation degree of the protein in the edible powder decreases incrementally going from the first to the second to the optionally third plurality. The kit of edible powders have a preset antigenicity which may advantageously be used to introduce or reintroduce protein into a diet of an individual in a controlled way via a stepwise increase of antigenicity levels over time. The kit of edible powders may advantageously be used to assist in the early stages of the milk ladder, and to standardize the baking process behind the milk ladder. Hence, according to a third aspect of the invention there is provided a kit of parts suitable for introducing or reintroducing protein to a human subject allergic to the protein or at risk of becoming allergic to the protein, the kit comprising:
(a) a plurality of a first edible, allergenic powder as described above, having a first reduced reproducible antigenicity;
   and preferably also comprising:
(b) a plurality of a second edible, allergenic powder, having a second reproducible antigenicity, and
(c) optionally a plurality of a third edible, allergenic powder having a further reproducible antigenicity;
   the first edible allergenic powder having a first reproducible, reduced antigenicity, and is suitable for being administered first; wherein the antigenicity of the second edible powder is higher than the antigenicity of the first edible powder, and the antigenicity of the optional third edible powder is higher than the antigenicity of the preceding edible powder. Preferably, the powders (a), (b) and (c) may be characterized in terms of the reproducible glycation degree which is linked to antigenicity; the first edible allergenic powder comprises protein having a first reproducible glycation degree, and is suitable for being administered first; wherein the glycation degree of the protein of the second edible powder is lower than the glycation degree of the protein of the first edible powder, and the glycation degree of the protein of the optional third edible powder is lower than the glycation degree of the protein of the preceding edible powder. The protein comprised in the powder is a glycated milk protein comprising beta-lactoglobulin (BLG), preferably BLG-A, for instance whey protein, including sweet or acid whey.

Associated with the above, there is provided a kit of parts for use in introducing or reintroducing protein to a human subject allergic to the protein or at risk of becoming allergic to the protein; and/or inducing oral immune tolerance to protein to a human subject allergic to the protein, and/or treating a human subject allergic to the protein, wherein the protein to which the human subject is allergic (or at risk) is introduced in time with incremental, predetermined antigenicity. Worded differently, the invention relates to a method for inducing oral immune tolerance to protein to a human subject allergic to the protein; and/or for treating a human subject allergic to the protein, wherein the protein to which the human subject is allergic (or at risk) is introduced or reintroduced in time with incremental, predetermined antigenicity using the kit of parts as described above. In other words, the invention also relates to the use of edible powders as described herein, in the manufacture of a kit of parts as described hereabove, for inducing oral immune tolerance to protein to a human subject allergic to the protein; and/or for treating a human subject allergic to the protein, wherein the protein to which the human subject is allergic (or at risk) is introduced or reintroduced in time with incremental, predetermined antigenicity as described above. For the children allergic to the protein, the powder aims to support oral tolerance development. For the child at risk of being allergic it provides a standardized alternative for introducing milk before introducing cow's milk itself.

It is preferred that the oral immunotherapy starts with administering a dosage of a first allergenic, edible powder A having a preset and reproducible antigenicity, followed by administering increased dosages of the first powder, preferably followed by administering increased dosages of a second allergenic, edible powder B with a preset and reproducible antigenicity which is higher than the antigenicity of powder A (following an administration regime involving e.g. A-AA-B-BB or A-AA-AAA-B-BB-BBB, provided that each consecutive administration step involves an antigenicity which is higher than that of the preceding step). It is preferred that the first allergenic, edible powder A is present in an amount which has a preset and reproducible antigenicity, and the preferred second allergenic, edible powder B is preferably present in an amount which has a preset and reproducible antigenicity that is higher than that of A, wherein the OIT starts by administering incremental dosages of the first, allergenic powder A, followed by administering incremental dosages of the second allergenic, edible powder B. It is preferred that the final edible powder is administered in an amount corresponding to a reproducible antigenicity of at most 50, preferably at most 20, more preferably at most 10, based on the aforementioned reference. The inventors found that the most important steps of the milk ladder are in the first parts (cookies, muffins), and noticed that the increase of antigenicity in the milk ladder is not so much linear but rather exponential. The new manufacturing allows for the use of powders with small antigenicity and allows for smal(ler) steps of allergenicity increase in the beginning, and which are administered with an antigenicity level which corresponds to those of steps 1-4 (cookies, muffins) in table 1 further on in the application. The skilled person will understand that the above regarding administering powders with incrementally higher antigenicity applies to administering powders with incrementally lower glycation degree of the protein in the edible powders. Consequently, the first allergenic, edible powder A has a reproducible and predetermined glycation degree and is administered first, wherein the second allergenic, edible powder B has a second reproducible and predetermined glycation degree which is lower than the glycation degree of A. Instead of preference for the final edible powder administered in an amount corresponding to a reproducible antigenicity of at most 50 based on the reference, the powder is preferably characterized in that the glycation degree of the powder that has the highest glycation degree is between 10 and 30% expressed as percentage of lysine residues blocked by the reducing sugar.

### Edible Powder Product and its Manufacture

It is manifest to provide a process of manufacturing a powder with protein with a predetermined and reproducible antigenicity. The antigenicity is determined using ELISA.

The manufacturing process requires that the powder contained in a heat-conductive pipe is heated over at least part of the length of the pipe (i.e. a ‘powder path’) to an extent that heating results in glycation of the proteins within the powder, and caking is prevented. Homogeneous heating of the powder is achieved by external vibration of the pipe. With homogeneous heating it is understood that all the powder heated in step (b) is subject to the same temperature and same amount of vibration. Glycation is achieved through reaction of the free amino groups of the proteins with the reducing sugars. Glycation itself is a phenomenon that is well-known to the skilled person in the art. The heating may be provided by heating the walls of the thermally conductive pipe, preferably a metal pipe.

A suitable heating technique for the dry edible powder is for example described in US2009/067470, although there it is particularly advocated for heating dry powders in the unrelated field of building industry. Its content is herewith incorporated by reference. The powder may be heated in a passage tube for current (flow) positioned tilted with respect to a horizontal plane, and preferably substantially vertically with respect to this plane, said powder flowing into said tubes essentially by gravity, and wherein said products are heated in said tube by heating the walls of the tube by the Joule effect, said tube being connected and directly powered by an electric power supply device with which the walls of said tube may be heated by the Joule effect. It is understood that the heating of the powder is essentially accomplished by radiation and, if necessary, by contact with the wall in the case of a tilted tube. In the context of the invention, the term ‘tube’, ‘channel’ and ‘pipe’ are used interchangeably, referring to a hollow cylindrical heat-conductive material allowing for flow of the edible powder while being heated. The manufacturing process may involve Revtech heat treatment technology that is commercially available from Revtech (Chicago; https://revtech-ps.com/food.html). The skilled person is provided with guidelines for operating the device for straightforwardly producing a powder with a reproducible antigenicity. It is preferred to use a 150 kg/h pilot unit with 8 spirals in a DN80 pipe.

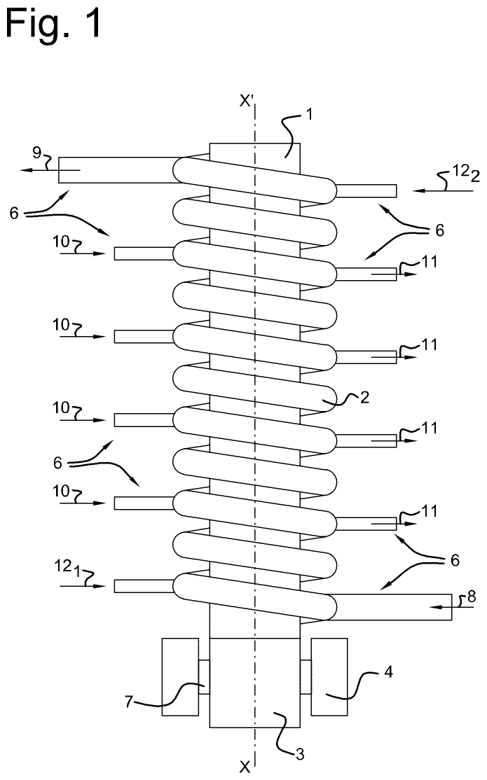

The heat may be continuous heating. The powder is preferably moved across the powder path preferably at a flow rate from 10 to 1000 kg/hour, preferably from 200 to 1000 kg/hour. The powder may be located within the pipe from 5 to 600 minutes. The powder may be heated at a temperature of at most 150° C., preferably from 60 to 150° C., more preferably from 70 to 120° C., even more preferably from 70 to 100° C. Flow rate can be adjusted to optimize the heating time. The appropriate combination of flow, heating time and temperature is considered to fall within the skilled person's conventional skills, with ELISA test being available for determining the actually achieved antigenicity as the product of the above parameters. An introduction device comprising a feed screw is advantageous because the feed rate of the product may be accurately controlled regardless of the applied gas flow rate. Advantageously, in particular if the tube is tilted, a gas is caused to flow with the current or against the current of the powder in said tube during the heating of the powder. An example is given in FIG. 1.

The pipe may have an internal diameter of from 2 to 100 cm, preferably from 10 to 80 cm, more preferably from 15 to 30 cm. The pipe may have a length of from 5 to 80 m, preferably from 10 to 60 m, more preferably from 15 to 30 m. The pipe may have a wall thickness of from 1 to 20 mm, preferably from 2 to 10 mm, more preferably from 2 to 6 mm. However, the thickness, length and diameter all not limiting the invention; the pipe dimensions are selected to achieve consistent and controllable heating conditions for the powder within the pipe.

In order to achieve homogeneous heating of the powder, the use of vibration to the heated pipe or tube is important. The powder movement thus achieved exposes the powder to homogeneous heating and glycation, and thus results in reproducible, uniform reduced antigenicity. The pipe may be vibrated through use of a vibrating device. For example, a vibrating base may be used to support the pipe and to vibrate the pipe. The operator may tune the vibration frequency and amplitude, and adapt the settings to powders with different properties, for example for processing barley seeds and fine dairy proteins or flour. Reference is made to FR2788336—its content herewith incorporated by reference—to understand the operation of devices that can be used with the process according to the invention.

Within the pipe, relative humidity is preferably (controlled) below 80%. In one embodiment, the relative humidity is controlled in the range of 30-70%, more preferably 30-60% RH. The inventors have found that relative humidity may be controlled by letting moist air escape the vibrating pipe of step (b). By letting moist air escape, caking was avoided. Preferably, relative humidity is controlled by letting moist air escape the vibrating pipe, preferably through a valve. In a preferred embodiment relative humidity in the vibrating pipe is controlled below 80% RH, preferably below 70% RH, more preferably below 60% RH, more preferably below 50% RH, even more preferably below 40%, even more preferably in the range of 10-40% RH, most preferably 10-30% RH.

In a non-limiting embodiment, wherein the pipe is a helical channel, and step (b) of the manufacturing process may involve continuous heat treatment of the powder moving inside at least one helical channel winding around a central barrel mounted on a vibrating base supporting the assembly, said assembly channel being heated to a given temperature over at least part of the path of the powder. An example is provided in FIG. 1 of FR2788336. FIG. 1 is a side view of a lifting device suitable for carrying out step (b) in the manufacturing method according to the invention with the various conduits and injection orifices and for extracting the gases to be conveyed in the device. In a known manner, the vibrating helical lifting device for the transport of bulk products comprises at least one ramp or closed helical channel 2 winding around a central shaft 1 and a vibrating base 3 supporting the assembly, said vibrating base 3 comprising at least two unbalanced motors 4 arranged diametrically with respect to the vertical axis XX′ of the cylindrical barrel central 1 and mounted on flanges 7 integral with said vibrating base 3. Said helical channel 5 is fixed on said central shaft 1 by spacers 5. Said helical tube 2, in the form of a coil with separate turns, may have a developed length which may vary between one meter and several hundred meters: the pulverulent product is introduced at one end 8 of said coil 2 towards the lower part of the barrel 1 and advances upward in said tube 2 under the effect of the vibrations generated by the vibrating base 3 until reaching the other end of said tube from which it is extracted by outlet 9. Preheated water vapor is continuously introduced along said helical channel 2 through at least one orifice 10 of said channel, opening into a part heated to a given temperature, greater than 100° C., and said water vapor is extracted by another orifice 11 located in this same heated part 13.

Preferably, the vibrating pipe of step (b) is not a fluidized bed chamber, and the process does not involve fluidized bed technology. Preferably, the powder provided in step (a) is inserted into the vibrating pipe of step (b) without inflation of moistened air which is far from the gist of controlling relative humidity.

In a preferred embodiment, the vibrating pipe of step (b) is an helical channel winding around a central barrel mounted on a vibrating base supporting the assembly, said assembly channel being heated to a given temperature over at least part of the path of the powder, wherein the helical channel is provided with inlet/outlet valves, wherein the relative humidity within the helical channel is controlled by letting moist air escape the valves. Preferably, relative humidity is controlled below 80%, preferably below 70%, more preferably below 60%, more preferably below 50%, even more preferably below 40%, even more preferably in the range of 10-40% RH, most preferably 10-30% RH. Preferably, the central barrel is substantially vertical with respect to an horizontal plane, wherein the powder is provided at the bottom of the helical channel and is moved upwards by vibration of the assembly channel. Reference is made to FIG. 1 herein.

In a preferred embodiment, the powder is firstly subjected to a pretreatment in which the powder is heated in the vibrating pipe, preferably at a temperature which is lower or equal to the temperature at which the powder is heated in step (b). This pretreatment step ensures that the powder is pre-dried and relative humidity during the main treatment is easily controlled, avoiding caking. During pre-treatment, moisture can be removed and the % RH reduced. In this embodiment, the process according to the invention comprises the steps of:
(a1) providing a powder comprising protein and reducing sugar, preferably wherein the powder comprises a milk protein comprising beta-lactoglobulin (BLG), preferably BLG-A, for instance whey protein, including sweet or acid whey;
(b1) heating the powder of step (a1) homogeneously within a heated, vibrating pipe, to obtain a pre-dried powder;
(a) providing the powder of step (a1) comprising protein and reducing sugar, preferably wherein the powder comprises a milk protein comprising beta-lactoglobulin (BLG), preferably BLG-A, for instance whey protein, including sweet or acid whey;
(b) heating the powder of step (a) homogeneously within a heated, vibrating pipe,
wherein the powder is heated at a temperature from 60 to 150° C., preferably from 70 to 120° C., more preferably from 70 to 100° C.,
wherein the relative humidity (RH) within the pipe is controlled below 80%,
to obtain a powder wherein the protein has been glycated with the reducing sugar;
(c) optionally adding any heat-sensitive components and/or bulking agents to the powder after step (b),
(d) packaging the powder,

Wherein the powder is heated in step (b1) at a temperature that is lower or equal to the temperature at which the powder is heated in step (b). Preferably, the powder is heated in step (b1) at a temperature of 40 to 120° C., more preferably from 60 to 100° C., even more preferably 70 to 90° C.

In one embodiment, the milk protein is whey protein.

The edible powder may be a whole milk powder, a reduced fat milk powder, a low-fat or skim milk powder, a fat-free milk powder, a buttermilk powder, yogurt powder or combinations thereof. The powder is preferably a cow's milk powder derived from cow's milk using conventional techniques. However, the milk of other animals, such as goats, may also be used. The powder may be a concentrate or an isolate.

The term protein according to the invention, in addition to its convention meaning, is taken to mean “protein component”. By way of example, whey proteins are not a single protein but consist of a number of individual protein components. The individual components in whey protein comprise beta-lactoglobulin, alpha-lactalbumin, lactoferrin, immunoglobulins, lactoperoxidase, bovine serum albumin (BSA), lysozyme and others, such as glycomacropeptide, which is only present in cheese whey. Put another way, the term protein may be taken to mean several different species of protein which are colloquially referred to by a generic name.

The amount of protein in the powder is preferably at least 1 wt %, more preferably at least 2 wt %, more preferably at least 5 wt % based on the total weight of the powder, preferably 5-50 wt %.

The powder also comprises a reducing sugar. The reducing sugar may conveniently be present in the protein source (e.g. milk powder comprises lactose) but—if so desired—a reducing sugar may also be added to the powder prior to (b) heating. The concept of a reducing sugar is known to the skilled person. The reducing sugar may be lactose, maltodextrin, or combinations thereof, preferably lactose. Preferably, maltodextrins with high dextrose equivalents (DE) are used. For example, maltotriose, maltotetraose, or combinations thereof may be used. In a preferred embodiment, the amount of reducing sugar in step (a) is at least 10 wt %, more preferably at least 20 wt %, most preferably 30-60 wt % of the powder.

Preferably, the weight ratio of reducing sugar to protein in the powder provided in step (a) is in the range of 10:1 to 10:12, more preferably 10:2 to 10:10, even more preferably 10:4 to 10:8. Preferably, the reducing sugar is lactose and the weight ratio of lactose to protein in the powder provided in step (a) is in the range of 10:1 to 10:12, more preferably 10:2 to 10:10, even more preferably 10:4 to 10:8.

The protein and the reducing sugar provided in step (a) react in a glycation reaction. Protein glycation refers to the non-enzymatic reaction between sugars and proteins. The main reaction is between the reducing sugar and the s-amino group of lysine residues. The glycation degree per each protein is defined herein as the percentage of lysine residues that are blocked by the reducing sugar. In a preferred embodiment, the protein is milk protein, for instance whey protein, including sweet or acid whey, wherein the milk protein comprises beta-lactoglobulin (BLG), preferably BLG-A.

In a preferred embodiment, the reducing sugar is lactose. Accordingly, the lactosylation degree of the milk protein is measured as the percentage of lysine residues blocked by lactose on BLG, preferably BLG-A. Preferably the lactosylated milk protein in the edible powder product of the invention has a lactosylation degree of at least 5%, preferably at least 10%, more preferably at least 15% most preferably 10-30% expressed as the percentage of lysine residues that are blocked by lactose on BLG, preferably BLG-A. In one embodiment, the lactosylated milk protein in the edible powder product of the invention has a lactosylation degree of at least 20%, expressed as the percentage of lysine residues that are blocked by lactose on BLG, preferably BLG-A.

The powders may be mixed into a liquid (drink) or mixed into or sprinkled onto solid food to form a nutritional composition. For example, the powder can be dissolved in water. The powder is added to a drink or food that does not contain the allergen. Also, the powders may be mixed with other additives (such as flavorings) either before or after the heat treatment. The edible powder may be subjected to further processing or handling at the consumer's end, but the consumer is preferably cautioned to refrain from substantive heat treatment, i.e. mixing the edible powder into a hot drink. The composition may be accompanied from instructions to mix the powder into drinks with a temperature below 40° C., and to avoid milk drinks or milk powder-based drinks.

### Application

The edible powder as described here above may be used for use in introducing or reintroducing protein to a human subject allergic to the protein or at risk of becoming allergic to the protein; and/or inducing oral immune tolerance to protein to a human subject allergic to the protein, and/or treating a human subject allergic to the protein, wherein the protein to which the human subject is allergic (or at risk) is introduced in time with incremental, predetermined and reproducible antigenicity, starting with administering a first amount of an allergenic, edible powder A with a reproducible reduced antigenicity, followed by administering increased dosages of the first powder (e.g. AA, AAA, . . . ), preferably followed by administering increased dosages of a second allergenic, edible powder B with a reproducible antigenicity which is higher than the antigenicity of powder A (e.g. B-BB-BBB . . . ), optionally followed by administering increased dosages of a third allergenic, edible powder C (e.g. C-CC-CCC) with a reproducible antigenicity which is higher than the antigenicity of powder B, and wherein the final edible powder is preferably administered in an amount corresponding to a reproducible antigenicity of at most 50, preferably at most 20, more preferably at most 10, based on the reference. The concept of subjecting the human to increasing antigenicity in a controlled powdered incremental manner, and particularly at the initial stages of OIT involving antigenicities below 10, based on the reference, is new in the art; antigenicity for the product ranges in existing baked milk challenges are not known. The skilled person will understand that the what is said above regarding administering powders with incrementally higher antigenicity applies to administering powders with incrementally lower glycation degree of the protein in the edible powders. Consequently, the first allergenic, edible powder A has a reproducible and predetermined glycation degree and is administered first, wherein the second allergenic, edible powder B has a second reproducible and predetermined glycation degree which is lower than the glycation degree of A. Instead of preference for the final edible powder administered in an amount corresponding to a reproducible antigenicity of at most 50 based on the reference, it is preferred that the glycation degree of the powder that has the highest glycation degree is between 10 and 30% expressed as percentage of lysine residues blocked by the reducing sugar.

In the context of the invention, human subjects ‘at risk of becoming allergic to the protein’ preferably means infants with a parental history of atopic eczema, allergic rhinitis or asthma in mother and/or father.

As described above, the reproducible antigenicity has allowed the inventors to develop a kit comprising a plurality of edible powder compositions with increasing, predetermined and reproducible levels of antigenicity, suitable for use in oral immunotherapy, i.e. for use in introducing or reintroducing protein to a human subject allergic to the protein or at risk of becoming allergic to the protein. The kit of edible powders have a preset antigenicity which may advantageously be used to introduce or reintroduce protein into a diet of an individual in a controlled way via a stepwise increase of antigenicity levels over time. The kit may comprise:
(a) a plurality of a first allergenic, edible powder A as described above, having a first reduced reproducible antigenicity;

In one embodiment, the kit may be accompanied with a program indicating the amount of individually packed first edible powder with a dose.

In a preferred embodiment, the kit also comprises:
(b) a plurality of a second allergenic edible powder B, having a second reproducible antigenicity, and optionally
(c) a plurality of a third allergenic, edible powder C having a further reproducible antigenicity;
   the first allergenic, edible powder having a first reproducible antigenicity, and is suitable for being administered first; and preferably the second and optionally third allergenic, edible powders having a reproducible antigenicity which is higher than the antigenicity in which the preceding edible powder is dosed. Preferably, the second and optionally third allergenic, edible powders comprise protein having a glycation degree which is lower than the glycation degree of the protein in the preceding edible powder. This is suited when limiting the amount of powders to be consumed and/or when the OIT involves an extended part of the milk ladder.

In a first embodiment, a plurality is preferably understood to mean that there is between 5 and 50, more preferably between 10 and 20 of individually packaged edible powders having the same reproducible antigenicity.

In an embodiment the kit comprises at least 5, preferably at least 10 packages of a first allergenic, edible powder A. In a preferred embodiment, the kit also comprises at least 5, preferably at least 10 packages of a second allergenic, edible powder B. Such a kit facilitates administering a single dose of A, then 2 dosages of A (AA), then 3 dosages of A (AAA), and optionally switching to a single dose of powder B, wherein the antigenicity of B is higher than that of the preceding antigenicity of the dosage of A, etc.,

In one embodiment, the kit may also comprise a plurality of packages of the first allergenic, edible powder having an increased dosage A′ and/or a plurality of packages of the second allergenic, edible powder having an increased dosage B′. By using different dosages combinations of A and A′ and/or B and B′, it may be possible to reach to increased antigenicities but limiting the amount of individual packages needed to achieve such antigenicities. This is both convenient in terms of handling, and increased the reliability (e.g. AAAAA vs. A+A′).

The edible powders are preferably packaged in cups, sachets or the like. Each package comprises one portion of edible powder. Preferably, the edible powders are individually seal packed, for example blister packed, for example when provided in compressed form. Sachets holding a single portion of edible powder are preferred. As indicated here above, the consumption of multiple portions of individually packaged edible powder per dose is anticipated.

The term “kit of parts” or “kit” may also be described as a set or a system. The compositions are preferably provided as a kit of parts by being enclosed in a single packaging (e.g. carton, box, net or foil) and/or (mutually) physically interconnected by means of connecting portion(s) which are preferably breakable. The kit of parts may be in the form of a wrap, a blister or container (e.g. a box) collectively holding the edible powders individually packaged as described here above. In one preferred embodiment, the kit of parts is a box with at a multitude of sachets, wherein each sachet contains a portion of edible powder. The kit may be accompanied by instructions indicating the order and sequence for administering the edible powders, for example using color-coding.

As said above, the invention also pertains to the kit as described above, for use in oral immunotherapy, i.e. for use in inducing oral immune tolerance to protein to a human subject allergic (or at risk thereof) to the protein; and/or for treating a human subject allergic (or at risk thereof) to the protein, wherein the protein to which the human subject is allergic (or at risk thereof) is introduced or reintroduced in time with incremental, predetermined antigenicity as described above. Worded differently, the invention relates to a method for inducing oral immune tolerance to protein to a human subject allergic (or at risk thereof) to the protein; and/or for treating a human subject allergic (or at risk thereof) to the protein, wherein the protein to which the human subject is allergic (or at risk thereof) is introduced or reintroduced in time with incremental, predetermined antigenicity as described above. In other words, the invention also relates to the use of the edible powders according to the invention in the manufacture of a kit as described above, for inducing oral immune tolerance to protein to a human subject allergic (or at risk thereof) to the protein; and/or for treating a human subject allergic (or at risk thereof) to the protein, wherein the protein to which the human subject is allergic (or at risk thereof) is introduced or reintroduced in time with incremental, predetermined antigenicity as described above. The subjects are thus exposed with time to the protein with incremental antigenicity. Worded differently, the invention relates to a method for inducing oral immune tolerance to protein to a human subject allergic (or at risk thereof) to the protein; and/or for treating a human subject allergic (or at risk thereof) to the protein, wherein the protein to which the human subject is allergic (or at risk thereof) is introduced or reintroduced in time with incremental, predetermined antigenicity.

The edible powders are orally administered.

It is preferred that the method is preceded by a step in which at least a first dose and optionally further dosages are consumed under medical supervision, in order to establish at which level the subject enters the program (which dose gives an issue, then the program goes back one step and the first step of the method starts with an antigenicity just below).

In an embodiment, the human suffers from protein allergy. In another embodiment the human is at risk of developing a protein allergy. The human subject may be an adult, infant or child, including toddlers. The human subject is preferably an infant or child, including a toddler, which is allergic to the protein, or at increased risk of becoming allergic to the protein. The children are preferably up to 12 years of age, preferably up to 6 years of age. The infant and toddlers are preferably 4 to 48 months of age, more preferably 4 to 36 months of age, even more preferably 4 to 24 months of age, most preferably 4 to 12 months of age. The subjects are preferably from 4 months up to 6 years of age. Providing edible powders which have a predetermined and reproducible level of antigenicity and a process that enables the production of protein powders with a predetermined and reproducible level of antigenicity allows for the introduction or reintroduction of protein to a human subject allergic to the protein or at risk of becoming allergic to the protein. The allergy is preferably egg, soy, nut, peanut or milk protein allergy, for example cow's milk protein allergy, most preferably BLG allergy.

In one embodiment, rather than using different powder A, B, C etc., the human may consume multiple portions of a powder having a specific antigenicity per portion, thus increasing the amount of portions per dose. However, in order to cover a more extended part of the milk ladder, the human may also consume combinations of portions of different powders.

The human subject may be exposed to an amount of a first edible powder A having a first antigenicity, followed by exposure to increased dosages of the first powder (e.g. AA, AAA, . . . ), preferably followed by exposure to increased dosages of a second allergenic, edible powder B with a reproducible antigenicity which is higher than the antigenicity of powder A (e.g. B-BB-BBB . . . ), optionally followed by exposure to increased dosages of a third allergenic, edible powder C (e.g. C-CC-CCC) with a reproducible antigenicity which is higher than the antigenicity of powder B, and wherein the subject is preferably exposed to an antigenicity that gradually increases from the first reproducible antigenicity, preferably starting with an antigenicity which corresponds to a cookie in the milk ladder. It is preferred that the OIT program timing between the consumption of the first and the last edible powder is between 2 and 24 months, more preferably between 3-12 months, preferably 3-6 months after the consumption of the first powder. The program allows for consumption of a dosage once a day, and compared to the conventional 3-times-a-week milk ladder therapy can be shortened to a great extent.

The human may participate in an OIT program with the frequency in which each edible powder is consumed can be such as described for conventional baked milk challenges in Kim et al. “Dietary baked-milk accelerates resolution of cow's milk allergy in children” J. Allergy Clin. Immunol. 2011; 128(1): 125-131, its contents herewith incorporated by reference. Edible powders with the same, reproducible antigenicity may be administered over days, weeks or months, preferably weeks, before increasing the dose. For example, an edible powder having a reproducible antigenicity may repeatedly be administered at least once per 1-3 days for 1-4 weeks, followed by administering the next edible powder at least once per 1-3 days for 1-4 weeks etc. it is preferred that the edible powders are administered at least once per day. However, it is also possible to achieve the same by repeatedly administering the edible powder having a reproducible antigenicity at least once per 1-3 days for 1-4 weeks, followed by administering the two (or more) portions of the edible powder at least once per 1-3 days for 1-4 weeks etc., each time increasing the amount of portions per dose. The program may also involve combinations of the two embodiments described above, provided that the allergenicity increases (stepwise) over time. Compared to conventional baked milk challenges and ladders, the therapy intensity may be increased since the format of the edible powders allows it to be fit into the human's daily routine straightforwardly, and the therapy according to the invention does not have the disadvantageous impact of existing baked milk challenges.

As used herein, unless otherwise expressly specified, all numbers such as those expressing values, ranges, amounts or percentages may be read as if prefaced by the word “about”, even if the term does not expressly appear. The term “about” when used herein means +/−10% of the stated value. Also, any numerical range recited herein is intended to include all sub-ranges subsumed therein. Singular encompasses plural and vice versa. Additionally, although the present invention has been described in terms of “comprising”, the process, powders and sets detailed herein may also be described as “consisting essentially of” or “consisting of”.

For a better understanding of the invention, and to show how embodiments of the same may be carried into effect, reference will now be made, by way of example, to the following experimental data.

### EXAMPLES

### Example 1: The Antigenicity of Products of the iMAP Milk Ladder

The data are taken from existing milk ladders, and the antigenicity was determined in order to design a kit of edible powders with increasing antigenicity. The antigenicity of existing milk ladder products was not known in the art.

The following information relates to a selection of baked milk foods that are used in a milk ladder. The foods are listed in table 1.

Entries 1 and 2 of Table 1 were prepared by mixing wheat or wheat-free flour (125 g), with xanthan gum (if wheat-free flour is used) (1 g) and skimmed or non-fat milk powder* (2 g). Cold dairy free spread (50 g) was then rubbed into the mixture. To the mixture was then added fruit (i.e. grated apple, grated pear or pureed banana) (31 g to 42 g) and vanilla (to taste) with further mixing. The mixture was then rolled out and cut into 20 finger sized strips. The strips were then baked at 180° C. for 10 to 15 minutes. Each portion (strip) contained 1 ml of milk and 0.3 g of milk protein. Entry 1 was preferably used initially and required the child to eat one cookie. Entry 2 was then subsequently used and required the child to eat three cookies.

Entries 3 and 4 of Table 1 were prepared by mixing wheat or wheat-free flour (250 g), with xanthan gum (if wheat-free flour is used) (3 g), baking powder (10 g), sugar (25 g) and salt (pinch). Sunflower oil or canola oil (50 ml) and milk** (250 ml) were then mixed together and added to the dry mixture with further mixing. Finely chopped or mashed apple, pear or banana (110 g) and vanilla (to taste) were then added to the aqueous mixture with further mixing. The mixture was then divided into 10 balls and baked at 180 to 200° C. for 15 to 20 minutes. Each portion (muffin) contained 25 ml of milk and 0.875 g of milk protein. Entry 3 was preferably used initially and required the child to eat half a muffin. Entry 4 was then subsequently used and required the child to eat one muffin.

Entries 5 and 6 of Table 1 were prepared by mixing together wheat or wheat-free flour (125 g), baking powder (10 g), salt (1 to 2 g), sunflower oil or canola oil (30 ml), milk** (250 ml) and water (50 ml). The mixture was then divided into 6 portions and fried in a hot pan. Each portion (pancake) contained 42 ml of milk and 1.47 g of milk protein. Entry 5 was preferably used initially and required the child to eat half a pancake. Entry 6 was then subsequently used and required the child to eat one pancake.

Entry 7 of Table 1 contained yogurt (125 ml). The yogurt has previously been heated to a temperature of 98° C. and no further heating was required. The yogurt was used as a single portion and contained 125 ml of milk and 6.0 g of milk protein.

Entry 8 of Table 1 contained UHT-treated skimmed cow's milk (100 ml; 3.47 g milk protein; Milbona AF Deutschland GmbH). The UHT-treated skimmed milk had previously been heated for 2-5 seconds at 140-150° C. The heat-treated skim milk was used as a single portion and contained 100 ml of milk and 3.47 g of milk protein.

The protein content of milk powder was calculated using conventional Dumas. The antigenicity was measured by ELISA assay. The values are expressed in percentages relative to the antigenicity of 100 ml UHT treated cow's milk Determination of anti-BLG-IgG-binding capacity by Enzyme-Linked Immunosorbent Assay (ELISA). Non-competitive ELISA was performed according to a method previously reported (Wang et al, Journal of Allergy and Clinical Immunology 2010, 125, 695-702). Briefly, the different milk protein samples were dissolved in Carbonate-Bicarbonate Buffer (Sigma) at a milk protein concentration of 5 μg/mL. A dilution range of BLG (Sigma), was used as a standard. Samples and standards were added to a 96 wells plate and incubated at 4° C. overnight to coat the wells with antigen. Plates were washed 4 times with PBS-Tween and blocked for 1 h with 1% gelatin in PBS at room temperature. Plates were washed and rabbit anti-BLG protein IgG antibody (Abcam, 1:10000 in PBS) was added and incubated for 1 h at room temperature. Plates were washed again. Peroxidase conjugated goat anti-rabbit IgG antibody (DAKO, 1:2000 in PBS) was added and incubated for 1 h at room temperature. Plates were washed, 1-step Ultra-TMB substrate (Thermo Fisher) was added, reaction was stopped by addition of 1M H2SO4 (VWR). The absorbance was measured at 450 nm (with 655 nm as reference filter).

TABLE 1
antigenicity determined for product range existing
baked milk challenge

| | Antigenicity (%)* of beta- |
|---|---|
| | lactoglobulin |
| 1. 1 × Cookie | 0.52 |
| 2. 3 × Cookies | 1.57 |
| 3. Half a Muffin | 0.60 |
| 4. 1 × Muffin | 1.20 |
| 5. Half a Pancake | 4.62 |
| 6. 1 × Pancake | 9.24 |
| 7. Yoghurt | 43.11 |
| 8. UHT treated skimmed milk | 100 |

*in percentages relative to the antigenicity of 100 ml UHT-treated cow's skim milk (Milbona AF Deutschland GmbH)

The results showed that the milk ladder increases in antigenicity when climbing the ladder. Therefore it formed a good reference for deciding the antigenicity levels that could be mimicked by the powder product. However as mentioned above in practice the use of the milk ladder itself has disadvantage mentioned above, that the antigenicity varies, for example based on small differences between individual ovens, and the use of some of the specific food ingredients may be a problem with fuzzy eaters. Table 1 therefore acts as a reference point for deciding suitable antigenicity levels for protein powders prepared by the process according to the invention.

### Example 2: Process of Preparing an Edible Powder According to the Invention

A skimmed milk powder comprising 36.8 wt % milk protein (N 6.38), 0.5 wt % fat, 3.7 wt % moisture, 7.8 wt % ash and 51.2 wt % lactose based on the total weight of the powder (OMIRA GmbH Ravensburg, Germany) was treated using a continuous heat treatment by direct contact heating whilst be moved by vibration at a flow rate of 140 kg/hr (i.e. transport by vibration). The heat treatment involved the RevTech heat treatment technology as described in US2009/067470 and was commercially available from Revtech (Chicago; https://revtech-ps.com/food.html), using a 150 kg/h pilot unit with 8 spirals in a DN80 pipe. The working conditions involved a relative humidity between 30 and 60%.

By way of example, three trials each comprising 15 kg of skimmed milk powder were treated using the process described above. In particular, the skimmed milk powder was subjected to a powder pre-treatment at 80° C. for 9 minutes through a pipe with a diameter of 8.5 cm and a length of 30 m. Here, the skimmed milk powder was continuously cycled through the pipe twice at 4.5 minutes per cycle. The skimmed milk powder was then subjected to a main powder treatment at 100° C. for up to 6 hours through a pipe with a diameter of 8.5 cm and a length of 30 m.

During the pre-treatment and main treatment samples (~100 g) were taken from each trial every 4.5 minutes and the antigenicity of beta-lactoglobulin was determined in the skimmed milk powder of trials 1, 2 and 3, according to the procedure described in Example 1. The results are shown in Table 2. The antigenicity was determined for these 100 g samples using the same assay as in example 1.

TABLE 2
reproducibility allergenicity

| | Anti-BLG | Anti-BLG | Anti-BLG | |
|---|---|---|---|---|
| Time point/ | binding | binding | binding | |
| temperature | Trial 1 | Trial 2 | Trial 3 | Average |
| 0 min, RT \hat{ } | 100 ± 2.44* | 93 ± 1.22 | 100 ± 9.09 | 97.7 ± 1.9 |
| 9 min 80° C., | 67 ± 1.83 | 68 ± 1.83 | 67 ± 2.52 | 67.3 ± 0.27 |
| 9 min 100° C. | | | | |
| 9 min 80° C., | 78 ± 1.83 | 77 ± 0.61 | 78 ± 2.54 | 77.7 ± 0.27 |
| 18 min 100° C. | | | | |
| UHT treated milk | 23.3 | | | |
| of example 1** | | | | |

*Set as the reference value in table 2, in order to determine reproducibility.
**The antigenicity of the pasteurized (20 seconds, 72° C.) milk protein in the powder [at timepoint 0] was compared with the antigenicity of the UHT milk of example 1 and was a factor 4.3 higher compared to the UHT milk protein. Hence, the numbers in table 2 can be compared to those of example 1 by multiplying the antigenicity numbers in table 2 with a factor 4.3.\hat{ } RT = 20° C.

Entry 1 of Table 2 shows the antigenicity of beta-lactoglobulin binding in trials 1 to 3 normalised to the antigenicity of the skimmed milk powder before starting the heat treatment (t=0, RT). Entry 2 of Table 2 shows the antigenicity of beta-lactoglobulin binding in trials 1 to 3 having undergone a pre-treatment for 9 minutes at a temperature of 80° C. and a main treatment for 9 minutes at a temperature of 100° C. Entry 3 of Table 2 shows the antigenicity of beta-lactoglobulin binding in trials 1 to 3 having undergone a pre-treatment for 9 minutes at a temperature of 80° C. and a main treatment for 18 minutes at a temperature of 100° C.

With the above information, it could for instance be calculated how much of the heat-treated powder of trial 2 (antigenicity of 67) would be needed to reach the same antigenicity in the milk ladder of example 1. This is plotted in table 3:

TABLE 3
amount of powder of trial 2 in table 2 needed to reach the same
antigenicity in the milk ladder of example 1

| | | Amount of protein of point | |
|---|---|---|---|
| | Antigenicity | 2 (9 min 80° C., 18 min | |
| | (%)* of | 100° C.) in example 2 needed | |
| | beta- | to come to same amount of | |
| | lactoglobulin | antigenicity in milk ladder | |
| 1. 1 × Cookie | 0.52 | 6 | mg |
| 2. 3 × Cookies | 1.57 | 19 | mg |
| 3. Half a Muffin | 0.60 | 7 | mg |
| 4. 1 × Muffin | 1.20 | 14 | mg |
| 5. Half a Pancake | 4.62 | 55 | mg |
| 6. 1 × Pancake | 9.24 | 111 | mg |
| 7. Yoghurt | 43.11 | 517 | mg |
| 8. 100 ml UHT | 100 | 1199 | mg |

skimmed milk (3.47
g protein)

| | |
|---|---|
| Revtech protein | 430 t = 0 |
| (reference in table 2) | 289.4 point 2 (67.3%) |
| 3.47 g | 334.1 point 3 (77.7%) |
| Revtech protein | 123.9 t = 0 |
| antigenicity | 83.4 point 2 |
| recalculated per g | 96.3 point 3 |
| protein | |

### Example 2a: Characterisation of the Edible Powder According to the Invention

Samples from each trial of Example 2 were analysed by MRM Mass Spectrometry as described here below.

### Materials

Analytical-grade formic acid (FA) was obtained from Honeywell Fluka (Roskilde, Denmark). LC-grade acetonitrile (ACN), LC-MS-grade water, ammonium formate, DL-LAN and L-Lysine-d4 were obtained from Sigma-Aldrich (St. Louis, MO, USA). DL-cystine-d4 was obtained from Cambridge Isotope Laboratories (Tewksbury, MA, USA). Analytical standards of LAL, CEL, CEL-d4, CML, CML-d4, furosine and furosine-d4 were from Iris Biotech GmbH (Marktredwitz, Germany). No commercial standard of histidinoalanine is available; therefore, it was not included.

### Acid Hydrolysis

Acid hydrolysis of proteins in milk or milk protein powder was conducted using reconstituted powder (100 mg protein mL-1 solution) mixed with 200 mL 10 M HCl in a glass vial and flushed with nitrogen to eliminate air. The samples were hydrolysed at 110° C. for 24 h. The acid hydrolysed samples were subsequently cooled to room temperature, 700 mL LC-MS grade water were added and centrifuged for 15 min at 14,000×g at 4° C. The supernatant were dried in a Genevac EZ-2 plus HCl speedvac at 40° C. (SP Scientific, United States) and reconstituted in a similar amount of LC-MS grade water (Sigma-Aldrich, St. Louis, MO, United States). Then, the sample was filtered through a 0.2 mM filter and the filtrate was mixed with LC-MS grade water (1:1, v/v) containing the internal standards at a concentration of 0.1 μg/mL of CEL-d4, CML-d2, and Lysine-d4, 0.2 μg/mL of Furosine-d4, and 1 μg/mL of Cystine-d4. (Cambridge Isotope Laboratories, Thaxted, UK) before the samples were injected into the LC-MS triple Q system. In total, the sample was diluted either 20 or 2000 times depending on the concentration in the sample.

### MRM Analysis

The acid-hydrolyzed samples were analyzed on an LC Infinity 1260 system coupled to a 6460 Triple Quad mass spectrometer (Agilent Technologies, Waldbronn, DE), which operated in MRM acquisition mode. Separation of the compounds released by acid hydrolysis was performed on an Intrada amino acid column (3 mm×150 mm, 3 μm, Imtrak). The mobile phases consisted of solvent A (100 mM ammonium formate) and solvent B (90% acetonitrile with 0.1% formic acid) using a flow rate of 0.6 mL/min. The column temperature was set to 50 C. The gradient was as follows: 40-100% A over 4 min, and held at 100% A for 5 min. The flow rate was increased to 0.8 mL/min over 1 min and held for 3 min; then, it returned to the initial condition over 1 min and was held for 4 min reequilibration. The injection volume was 2 μL. Quantification was calculated based on the ratio of the analyte and internal standard with MassHunter Quantitative Analysis software (Agilent Technologies).

### Results

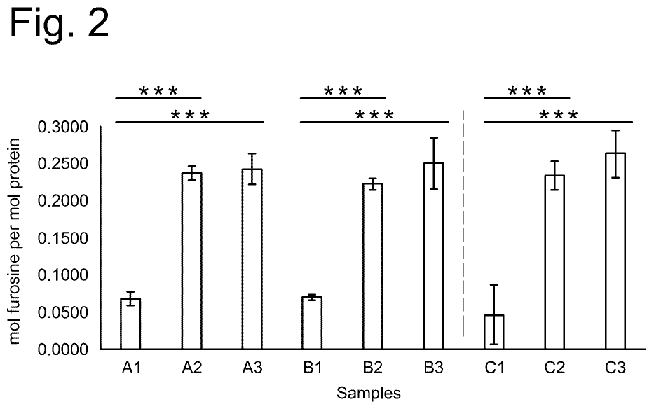

Results are reported in Tables 4-5 and FIGS. 2-3.

TABLE 4
Furosine formation

| Time point/ | Furosine (mol/mol | |
|---|---|---|
| temperature | protein) mean | Std |
| 0 min, RT \hat{ } Trial 1 | 0.0679 | 0.0095 |
| 0 min, RT \hat{ } Trial 2 | 0.0700 | 0.0037 |
| 0 min, RT \hat{ } Trial 3 | 0.0461 | 0.0400 |
| 9 min 80° C., | 0.2374 | 0.0095 |
| 9 min 100° C. Trial 1 | | |
| 9 min 80° C., | 0.2230 | 0.0079 |
| 9 min 100° C. Trial 2 | | |
| 9 min 80° C., | 0.2342 | 0.0190 |
| 9 min 100° C. Trial 3 | | |
| 9 min 80° C., | 0.2432 | 0.0212 |
| 18 min 100° C. Trial 1 | | |
| 9 min 80° C., | 0.2507 | 0.0349 |
| 18 min 100° C. Trial 2 | | |
| 9 min 80° C., | 0.2636 | 0.0319 |
| 18 min 100° C. Trial 3 | | |

Table 4 shows the advancement of the Maillard reaction by measuring Furosine which is an indicator of the formation of the Amadori product. Furosine increases in the powders treated according to the invention compared to the untreated powders. There are only small variations among Trials 1-3 which are within the std. The same is shown graphically in FIG. 2, wherein A1, B1, and C1 correspond to trials 1-3 untreated, A2, B2 and C2 correspond to trials 1-3 of 9 min treatment at 100° C. and A3, B3 and C3 correspond to 18 min treatment at 100° C. FIG. 2 shows low levels of furosine in the untreated samples (A1, B1 and C1). Furosine increases with treatment time and is lower in the powders treated for 9 minutes (A2, B2, C2) than in the powders treated for 18 minutes (A3, B3, C3). Very small variation are observed among A2-C2 and A3-C3, confirming that the level of Maillardation is reproducible.

TABLE 5
lactosylation of BLG-A

| | Lactosylation degree (% of |
|---|---|
| | lysine blocked by lactose) |
| Time point/temperature | Beta-lactoglobulin A |
| 0 min, RT \hat{ } Trial 1 | 6.2 |
| 0 min, RT \hat{ } Trial 2 | 7.1 |
| 0 min, RT \hat{ } Trial 3 | 6.5 |
| Average | 6.6 |
| 9 min 80° C., | 24 |
| 9 min 100° C. Trial 1 | |
| 9 min 80° C., | 23 |
| 9 min 100° C. Trial 2 | |
| 9 min 80 ° C., | 26 |
| 9 min 100° C. Trial 3 | |
| Average | 24 |
| 9 min 80° C., | 26 |
| 18 min 100° C. Trial 1 | |
| 9 min 80° C., | 24 |
| 18 min 100° C. Trial 2 | |
| 9 min 80° C., | 26 |
| 18 min 100° C. Trial 3 | |
| Average | 25 |

Table 5 and FIG. 3 show the results of protein lactosylation following the process of the invention. Beta-lactoglobulin is one of the main allergen in milk. BLG-A detection techniques for lactosylation are more reliable than compared to other milk proteins. Hence, BLG-A is chosen as representative of lactosylation on milk proteins. Untreated samples were mainly composed of proteins with 0 or 1 lactosylation sites (FIG. 3A). The treatment at 9 minutes yielded up to 6 lactosylation sites per protein, while the treatment at 18 minutes yielded up to 7 lactosylation sites per protein (FIG. 3A). The untreated samples show almost absent lactosylation of BLG-A with an average of 6.6% lysine residues on BLG-A blocked by lactose. After treatment for 9 minutes or 18 minutes, the lactosylation per protein increases to an average of 24% and 25%, respectively (Table 5 and FIG. 3B). Lactosylation of beta-lactoglobulin indicates lower antigenicity compared to the untreated sample. Small or no variation is observed among the three Trials 1-3 at 9 or 18 minutes, indicating reproducibility of the lactosylation process. Similar observations were made for BLG-B (data not shown).

The results reported in Tables 4-5 and FIGS. 2-3 indicate that the Maillard reaction-induced protein glycation is highly reproducible in the process of the invention.

### Example 3a: A Kit According to the Invention

There is provided a carton pack comprising at least 10 sachets of a milk protein powder with an antigenicity of 0.5, based on the UHT treated skimmed milk reference, and instructions for mixing the powder with a food or drink.

### Example 3b: A Kit According to the Invention

There is provided a carton pack comprising at least 5 sachets of a first milk protein powder present in an amount corresponding to an antigenicity of 0.5, based on the UHT treated skimmed milk reference; at least 5 sachets of a second milk protein powder present in an amount corresponding to an antigenicity of 1.0; based on the UHT treated skimmed milk reference and instructions for mixing the powders with a food or drink.

### Example 3c: A Kit According to the Invention

There is provided a carton pack comprising at least 10 sachets of a first egg protein powder with an antigenicity of 0.5, based on the antigenicity of 100 g of standard raw egg reference; 10 sachets of a second egg protein powder with an antigenicity of 1.0; and instructions for mixing the powders with a food or drink.

1.-13. (canceled)

14. A process of preparing an allergenic, edible powder product suitable for introducing or reintroducing protein to a human subject allergic to the protein or at risk of becoming allergic to the protein, the process comprising:
(a) providing a powder comprising protein and reducing sugar, wherein the protein in the powder is a milk protein comprising beta-lactoglobulin (BLG); and
(b) heating the powder of step (a) homogeneously within a heated, vibrating pipe,
wherein the powder is heated at a temperature from 60 to 150° C.,
wherein the relative humidity (RH-u) within the pipe is controlled below 80%,
to obtain a powder wherein the protein has been glycated with the reducing sugar;
(c) optionally adding any heat-sensitive components and/or bulking agents to the powder after step (b),
(d) packaging the powder,
to obtain a packaged amount of an allergenic, edible powder:
(i) having a predetermined antigenicity reduced at least 20% compared to the protein reference, wherein the protein reference is 100 ml UHT-treated cow's milk; and/or
(ii) wherein the glycated protein has a predetermined degree of glycation of at least 10% expressed as the percentage of lysine residues that are blocked by the reducing sugar on BLG,
wherein the antigenicity is determined using Enzyme-Linked Immunosorbent Assay (ELISA),

15. The process according to claim 14, wherein the powder is characterized by at least (ii).

16. The process according to claim 14, wherein the pipe is a helical channel, and (b) heating involves continuous heat treatment of the powder moving inside at least one helical channel winding around a central barrel mounted on a vibrating base supporting the assembly, said assembly channel being heated to a given temperature over at least part of the path of the powder.

17. The process according to claim 14, (b) heating the powder of step (a) homogeneously within a heated, vibrating pipe, wherein the powder is heated at a temperature from 70 to 120° C.

18. The process according to claim 14, wherein the milk protein comprises whey protein, including sweet or acid whey.

19. The process according to claim 14, wherein the powder provided in (a) is whole milk powder, a reduced fat milk powder, a low-fat or skim milk powder, a fat-free milk powder, a buttermilk powder, yogurt powder or combinations thereof.

20. The process according to claim 14, wherein the reducing sugar is lactose, maltodextrin, or combination thereof.

21. The process according to claim 14, wherein the weight ratio of reducing sugar to protein in the powder provided in step (a) is in the range of 10:1 to 10:12.

22. The process according to claim 14, wherein the weight ratio of reducing sugar to protein in the powder provided in step (a) is in the range of 10:2 to 10:10.

23. A kit of parts comprising:
(a) a plurality of a first allergenic, edible powder comprising protein and reducing sugar,
wherein the protein in the powder is a glycated milk protein comprising beta-lactoglobulin (BLG), obtainable by the process according to claim 14, having a first reduced reproducible antigenicity and/or a first reproducible glycation degree of the milk protein,
wherein (i) the antigenicity is reduced at least 20% compared to the protein reference,
wherein the protein reference is 100 ml UHT-treated cow's milk; and/or;
wherein (ii) the glycated milk protein has a glycation degree of at least 10% expressed as the percentage of lysine residues that are blocked by a reducing sugar on BLG-A,
wherein the antigenicity is determined using Enzyme-Linked Immunosorbent Assay (ELISA),

24. The kit of parts according to claim 23, wherein the powder is characterized by at least (ii).

25. The kit according to claim 23, accompanied from guidelines indicating the amount of individually packed first edible powder in a dose.

26. The kit according to claim 23, further comprising:
(b) a plurality of a second allergenic, edible powder comprising protein and reducing sugar, wherein the protein in the powder is a glycated milk protein comprising beta-lactoglobulin (BLG), obtainable by the process according to claim 1, having a second reproducible antigenicity, and optionally
(c) a plurality of a third allergenic, edible powder comprising protein and reducing sugar, wherein the protein in the powder is a glycated milk protein comprising beta-lactoglobulin (BLG), obtainable by the process according to claim 1, having a further reproducible antigenicity;
the first allergenic, edible powder having a first reproducible antigenicity, and is suitable for being administered first; and the second and optionally third allergenic, edible powders having a reproducible antigenicity which is higher than the antigenicity in which the preceding edible powder is dosed.

27. The kit according to claim 23, further comprising:
(b) a plurality of a second allergenic, edible powder comprising protein and reducing sugar, wherein the protein in the powder is a glycated milk protein comprising beta-lactoglobulin (BLG), obtainable by the process according to claim 1, and having a second reproducible glycation degree of the glycated milk protein, and optionally (c) a plurality of a third allergenic, edible powder comprising protein and reducing sugar, wherein the protein in the powder is a glycated milk protein comprising beta-lactoglobulin (BLG), obtainable by the process according to claim 1, and having a further reproducible glycation degree of the glycated milk protein;
the first allergenic, edible powder having a first reproducible glycation degree of the glycated milk protein, and is suitable for being administered first; and the second and optionally third allergenic, edible powders having a reproducible glycation degree of glycated milk protein which is lower than the glycation degree of the glycated milk protein of the preceding edible powder.

28. A method for introducing or reintroducing protein in a human subject allergic to the protein or at risk of becoming allergic to the protein, the method comprising administering to a human subject an allergenic, edible powder comprising protein and reducing sugar, wherein the protein in the powder is a glycated milk protein comprising beta-lactoglobulin (BLG), obtainable by the process according to claim 14, wherein the protein is cow's milk protein and the human subject is allergic or at risk of becoming allergic to cow's milk protein.
